(19) United States
(12) Plant Patent Application Publication
(10) Pub. No.: US 2014/0130216 P1
(43) Pub. Date: May 8, 2014

(54) Hybrid rose plant named 'IRB0182G'

(50) ;

(71) Applicant: Laura Rose BRIGHT, Richmond, IN (US)

(72) Inventor: Laura Rose BRIGHT, Richmond, IN (US)

(21) Appl. No.: 13/694,196

(22) Filed: Nov. 6, 2012

(51) Int. Cl. A01H 5/00

(52) U.S. Cl. PLT/134

(57) ABSTRACT

A new and distinct variety of hybrid rose plant named ‘IRB0182G’ characterized by its upright, freely branching growth habit that will produce basal breaks and branches from pinched plants, yellow inner petal color, and yellow-green outer petal and closed petal color.

### LATIN NAME OF THE GENUS AND SPECIES OF THE PLANT CLAIMED

Rosa sp. hybrid Tea Rose

### VARIETY DENOMINATION

‘IRB0182G’

### BACKGROUND OF THE INVENTION

The present invention relates to a new and distinct hybrid cultivar of Rose plant, botanically known as Rosa sp., hereinafter referred to by the cultivar name ‘IRB0182G’.

The new hybrid Rose ‘IRB0182G’ is a product of a breeding program conducted by the inventor, Laura Rose Bright, in Lasso, Ecuador. Asexual reproduction of the new hybrid rose ‘IRB0182G’ is performed by grafting. Propagation through patch budding has demonstrated that the combination of characteristics as herein disclosed for the new cultivar are firmly fixed and retained through successive generations of asexual reproduction. The new cultivar reproduces true-to-type.

### BRIEF DESCRIPTION OF THE PHOTOGRAPHS

The accompanying photographs illustrate the overall appearance of the new hybrid rose ‘IRB0182G’ showing the colors as true as is reasonably possible with colored reproductions of this type. Colors in the photograph may differ slightly from the color values cited in the detailed botanical description which accurately describes the color of ‘IRB0182G’.

FIG. 1 shows a flower, sepals, and foliage of ‘IRB0182G’.

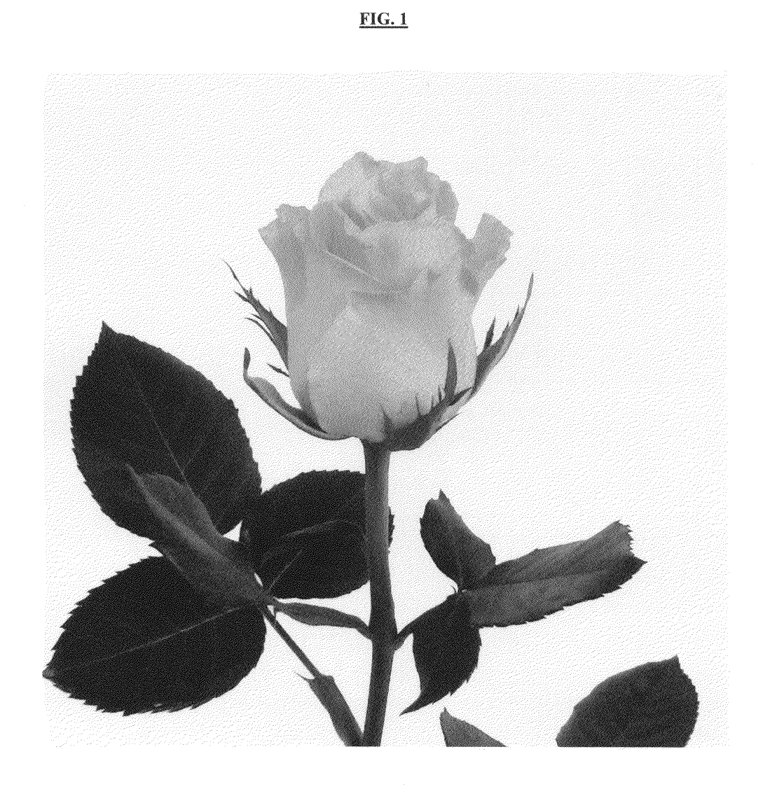

FIG. 2 shows a close-up view of a flower of ‘IRB0182G’.

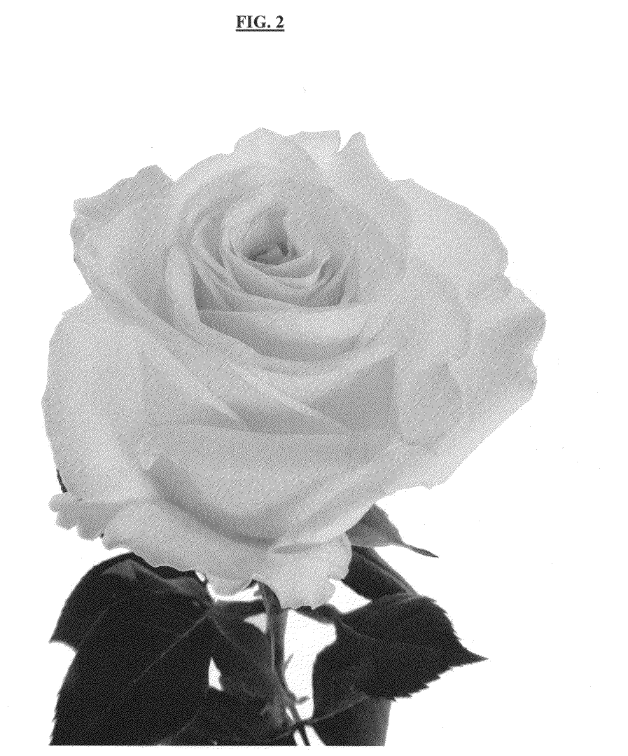

FIG. 3 shows a close-up view of the sepals of ‘IRB0182G’.

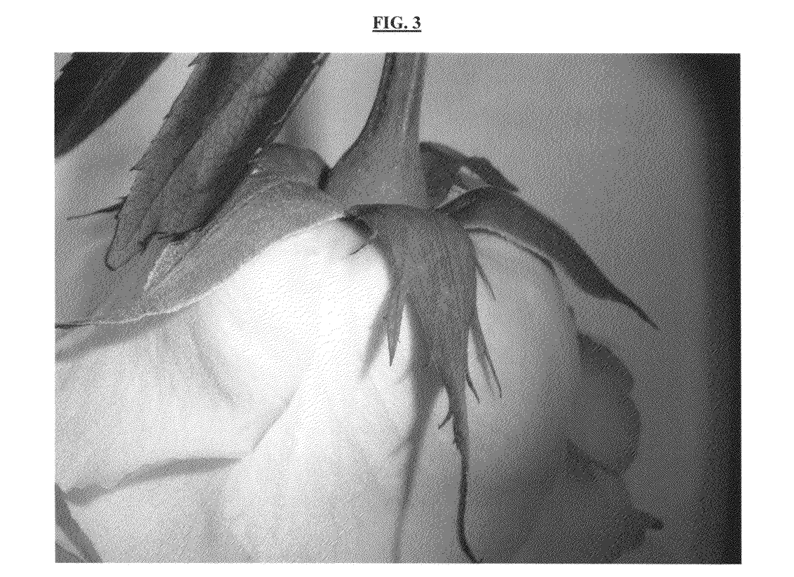

FIG. 4 shows a petiole of ‘IRB0182G’ with three leaflets.

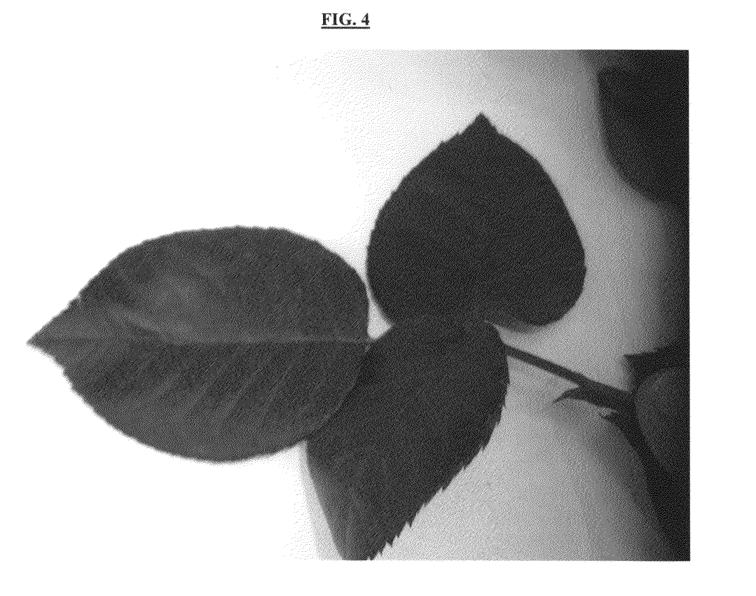

FIG. 5 shows a petiole of ‘IRB0182G’ with five leaflets.

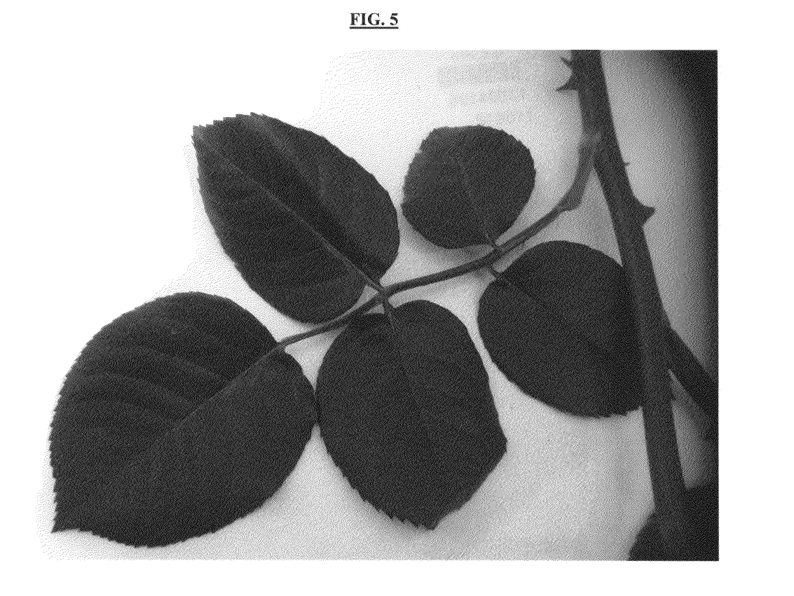

### DETAILED BOTANICAL DESCRIPTION

Plants of the cultivar ‘IRB0182G’ have not been observed under all possible environmental conditions. The phenotype may vary somewhat with variations in environment such as temperature and light intensity which can vary by season, without, however, any variance in genotype. The photographs and following observations and measurements describe plants grown in Lasso, Ecuador, under commercial practice in a polyethylene plastic covered greenhouse in a climate where the night temperatures may drop as low as 35 F but typically reach about 45 F and night and may rise to around 86 F but typically reach around 70 F day temperatures. The location is nearly 10,000 feet above sea level. The observations are from plants over one year old that were propagated by patch budding on a Natal Brier rose rootstock.
Botanical classification: Rosa sp. hybrid tea rose
Parentage:
   Female parent.—Proprietary Rosa sp. selection identified as code number19-85W, a white seedling, not patented.
   Male parent.—Proprietary Rosa sp. selection identified as code number92-177Y a yellow seedling, not patented.
Propagation: Although the cultivar can be propagated from a rooted cutting, it is recommended that it be budded on a suitable rootstock such as Rosa sp. var. Manetti or Natal Brier for greenhouse production of roses. The grafting can be as a bud, t graft stentling or other type of graft. The rootstocks for budded plants should generally be grown for several weeks if the grafting is in the greenhouse for the plant to be actively growing to accept the bud.
   Rooting habit.—Will depend upon the rootstock used.
Plant:
   General appearance.—The cultivar IRB0182G is a upright, freely branching grower that will produce both basal breaks and branches from the pinched plants.
   Plant height.—Typically, the plant will be managed with the flower cutting points between 30 cm and 150 cm.
   Plant diameter.—The plant will generally be managed with a diameter up to 30 cm.
   Internode length.—On stem, about 6 to 7 cm.
   Thorns.—Reddish brown color, up to about 12 cm from the base of the flower. The thorns under normal growing conditions are about 1 cm long.
   Leaves.—Generally with five leaflets, and upper two leaves generally have three leaflets, with the internode length near the top at about 5 cm. Color is generally similar to The Royal Horticultural Society Yellow Green group color 147A on the top of the leaves. The foliage is semi-glossy. The pinnate leaves have a slightly saw toothed edge and an oval shape.
   Cut flowers.—The cut flowers will generally have a stem length from 40 cm to 80 cm depending upon growing conditions, if the cutting is going up or down on the plant, etc.
   Flower size.—Flower bud height generally from 5.5 to 6.0 cm. Open flower diameter generally from 9 to 10 cm.
   Shape.—Inner rows of petals form comparatively flat surface area with the outer rows of petals at a progressively lower level.
   Color.—Inner petal color of open flowers is generally similar to The Royal Horticultural Society Yellow Group 11B color. The predominant color of the outer petals and top of petals of closed flowers is generally similar to The Royal Horticultural Society Yellow-Green Group 145A. Under colder growing condition or slightly lower light conditions, the green may be intensified. The color is greener in closed than open flowers.
   Petals.—The edge of the outer petals is slightly undulating, while the inner petal shape comes to a point.
   Sepals.—The sepals are narrow near the tip, approximately 4 cm long under normal conditions, and around six pairs of leaflike extensions on each side.
   Peduncles.—Peduncle is green like the rest of the stem.
Reproductive organs: None observed
Seed/fruit: None observed
Disease and pest resistance: Normal, with some susceptibility to Botrytis cinerea.

What is claimed is:

1. A new and distinct variety of hybrid rose plant named ‘IRB0182G’, as herein illustrated and described by the characteristics set forth above.
